(19) United States
(12) Patent Application Publication
(10) Pub. No.: US 2001/0039373 A1
(43) Pub. Date: Nov. 8, 2001

(54) Method of managing pharmaceutical dosage

(76) Inventors: Scott D. Cunningham, Chadds Ford, PA (US); Robert R. Dorsch, Hockessin, DE (US)

Correspondence Address: E I DU PONT DE NEMOURS AND COMPANY, LEGAL DEPARTMENT - PATENTS, 1007 MARKET STREET, WILMINGTON, DE, 19898, US

(21) Appl. No.: 09/823,529

(22) Filed: Mar. 30, 2001

Related U.S. Application Data
(60) Provisional application No. 60/193,983, filed on Mar. 31, 2000.

(51) Int. Cl. A61B 5/00

(52) U.S. Cl. 600/300

(57) ABSTRACT

A method is disclosed for the management of pharmaceutical dosage. The method involves transmitting medical data from a patient monitoring station via an electronic link to a centralized processing station where the data is analyzed and used to adjust pharmaceutical dosage which is provided to the patient in a personalized dosage delivery system. Optionally, analyzed data may be further enhanced at a data enhancement station where analyzed data from a multiplicity of central processing stations is accumulated and processed. Enhanced data may then be used to further refine the pharmaceutical dosage, alter the pharmaceutical being used, or develop new findings about the metabolic effects of the pharmaceutical.

This application claims the benefit of U.S. Provisional Application No. 60/193,983, filed Mar. 31, 2000.

### FIELD OF THE INVENTION

The invention relates to electronic monitoring systems for remotely managing pharmaceutical dosage in a patient. Specifically, a method has been developed which allows for the monitoring of patient physiological condition and adjusting the dosage of a pharmaceutical composition via remote electronic means.

### BACKGROUND OF THE INVENTION

Generally, telemedicine is a term used to describe a type of patient care which involves monitoring of a patient's condition by a healthcare worker located at a healthcare facility which is remote with respect to the location of the patient. Telemedicine, if adequately employed, is capable of providing enormous benefits to society. One such benefit is that patients can be examined without having to travel to a healthcare facility. This feature is particularly important for patients who live in remote areas who may not be able to easily travel to the nearest healthcare facility, or who need to be examined by a healthcare worker located far away from the patient, in another state, for example.

Another benefit of telemedicine is that it is capable of allowing a patient to be examined more often than would be possible if the patient were required to travel to a healthcare facility due to the ease with which it can be administered. For example, if a patient's condition requires that measurements be taken several times a day, it would be impractical for the patient to travel to and from a healthcare facility each time a measurement needs to be taken. It probably would be necessary for the patient to be admitted to the healthcare facility. The use of telemedicine could allow these measurements to be taken at the patient's home while the healthcare worker observed the patient or the measurement data from the healthcare facility.

Another benefit of telemedicine is that it allows a patient to be examined in a more timely manner than if the patient was required to travel to the healthcare facility. This is important in urgent situations, such as when a patient's condition becomes critical and emergency procedures must be taken immediately.

Various types of telemedicine systems are known. One example of such a system is disclosed in U.S. Pat. No. 5,441,047 (hereinafter '047), issued Aug. 15, 1995, which discloses an ambulatory patient health monitoring system for monitoring a remotely-located healthcare patient from a central station. The system includes instruments at the remote location for measuring the medical condition of the patient. The medical condition may correspond to health parameters, such as heart rate, respiratory rate, pulse oximetry and blood pressure. The system includes a first audiovisual camera disposed at the patient location and a second audio-visual camera disposed at the central station. Audio and video information is transmitted between the patient's remote location and the central station via a communications network, such as an interactive cable television network. Patient data is transmitted between the patient remote location and the central station by a separate communications network, such as satellite, radio transmission or telephone lines. A display is located at the patient's remote location and at the central station to allow the patient and the healthcare worker to observe each other simultaneously.

One of the disadvantages of the system disclosed in the '047 patent is that, although it refers to sending the information between the healthcare worker and the patient via various types of networks, the patent does not discuss using an algorithm for analysis of the information. Another disadvantage of the system disclosed in the '047 patent is that it does not mention using the analyzed data to adjust pharmaceutical dosage of a patient.

Another example of a telemedicine system is disclosed in U.S. Pat. No. 5,434,611 (hereinafter '611), issued Jul. 18, 1995. This patent discloses a telemedicine system having a two-way CATV network for transmitting images, voice and data between equipment located at the patient's home and equipment located at a medical office. Cameras are located in both the patient's home and in the medical office to provide return images between the doctor and the patient. In order for the doctor's terminal to communicate with the patient's terminal, the doctor's terminal sends a signal over a control line to the patient's terminal. A line controller then selects a communication channel for the session by selecting an unused channel in a multiple channel access (MCA) system. The terminals then automatically tune to the assigned communications channel and the information is communicated over the assigned channel between the patient and the doctor.

One disadvantage of the system disclosed in the '611 patent is that any communication between the doctor and patient must be set up by sending a signal which the line controller detects. The line controller then selects an unused channel for the communication. It also appears that the signal must be initiated by the doctor because the text of the patent only describes the situation where the doctor sends the signal to initiate the session. In any event, the system requires that a direct connection be made between the patient's terminal and the doctor's terminal. No provision is made for allowing medical measurement data to be sent to the doctor's terminal without a direct connection being made between the patient's terminal and the doctor's terminal. Therefore, in accordance with the system disclosed in the '611 patent, it would be impossible for information relating to the patient's condition to be sent by the patient's terminal to the doctor's terminal in the absence of a direct connection being made between the terminals, which requires that the doctor be present for the session.

It would be advantageous to provide a telemedicine system which would allow either a patient or a healthcare worker to initiate a diagnostic session to cause diagnostic measurements to be taken and sent to a location, such as a centralized processing station, where medical files could be automatically updated by the data. One advantage of such a system is that a healthcare worker would not have to administer a diagnostic session and, therefore, would not have to participate in the session. Another advantage of such a system is that medical files could be automatically updated without any action on the part of a healthcare worker being required. Furthermore, as the medical files are automatically updated, the patient's condition could be automatically monitored so that, in the event that the patient's condition falls below a predetermined level, remedial measures can be taken. It would also be advantageous to provide a telemedicine system which would allow patient information to further refine the pharmaceutical dosage, alter the pharmaceutical being used, or develop new findings about the metabolic effects of the pharmaceutical.

The problem to be solved, therefore is to provide a method for remotely managing pharmaceutical dosage in a patient. Applicants have solved the stated problem by developing a method which allows for monitoring a patient's physiological condition. In addition, a method has been developed which allows for adjusting the dosage of a pharmaceutical composition via remote electronic means. The method involves transmitting medical data from a patient monitoring station via an electronic link to a centralized processing station where the data is analyzed and used to adjust pharmaceutical dosage.

### SUMMARY OF THE INVENTION

The present invention provides a pharmaceutical monitoring and review system for transmitting medical data between a patient monitoring station and a central processing station and between the central processing station and one or more data enhancement stations over an integrated network the monitoring and review system comprising:

a) a patient monitoring station comprising a diagnostic means for measuring patient physiological condition in response to the presence of at least one pharmaceutical composition, the diagnostic means collecting and transmitting medical data via an electronic link means to a central processing station;

b) a central processing station for receiving medical data from the diagnostic means; the central processing station comprising an algorithm for analysis of the medical data and a data storage unit for the storage and retrieval of medical data; the central processing station transmitting the analyzed data to the patient monitoring station and optionally to at least one data enhancement station; and

c) a data enhancement station comprising at least one computer to receive the analyzed medical data from one or more central processing stations; the computer comparing the analyzed medical data received from the one or more central processing stations with prior generated data to generate enhanced data.

In a preferred embodiment the pharmaceutical monitoring and review system optionally provides a personalized pharmaceutical delivery system having a pharmaceutical contained within a non-toxic, biodegradable pharmaceutical delivery medium; wherein the pharmaceutical is distributed uniformly within the pharmaceutical delivery medium such that division of the delivery medium according to unit length or unit area may be used to quantify the dosage of the pharmaceutical.

In an alternate embodiment the invention provides a method of monitoring and reviewing the administration of a pharmaceutical composition by operating a pharmaceutical monitoring and review system of the present invention comprising:

a) collecting medical data in a diagnostic means at the patient monitoring station, the data reflective of a patient's physiological condition in response to the presence of at least one pharmaceutical composition;

b) transmitting the medical data from the diagnostic means via an electronic link means to a central processing station comprising an algorithm and a data storage unit for data analysis;

c) analyzing the medical data by the algorithm to generate analyzed data;

d) transmitting the analyzed data to least one computer located at a data enhancement station and to the patient monitoring station, whereby the dosage of the pharmaceutical composition is administered at the patient monitoring station;

e) collecting analyzed data from one or more central processing stations in the computer at the data enhancement station whereby enhanced data is generated; and

f) transmitting some or all of the enhanced data generated by the at least one computer to either or both of the patient monitoring station and the central processing station whereby dosage of the pharmaceutical composition is administered at the patient monitoring station.

In one preferred embodiment the invention provides a pharmaceutical management system for transmitting medical data between a patient monitoring station and a central processing station over an integrated network the management system comprising:

a) a patient monitoring station comprising a diagnostic means for measuring patient physiological condition in response to the presence of at least one pharmaceutical composition; the composition contained in a personalized pharmaceutical delivery system, the diagnostic means collecting and transmitting medical data via an electronic link means to a central processing station;

b) a central processing station for receiving medical data from the diagnostic means; the central processing station comprising an algorithm and a data storage unit for storage and analysis of the medical data; the central processing station transmitting the analyzed medical data to the patient monitoring station whereby the at least one pharmaceutical composition is administered according to the personalized pharmaceutical delivery system.

Additionally the invention provides a method for managing pharmaceutical dosage of a pharmaceutical contained in a personalized pharmaceutical delivery system by operating the pharmaceutical management system of the present invention comprising:

a) collecting medical data in a diagnostic means at the patient monitoring station, the data reflective of a patient's physiological condition in response to the presence of at least one pharmaceutical composition contained within a personalized pharmaceutical delivery system;

b) transmitting the medical data from the diagnostic means via an electronic link means to a central processing station comprising an algorithm and a data storage unit for data storage and analysis;

c) analyzing the medical data by the algorithm to generate analyzed data; and

d) transmitting the analyzed data to the patient monitoring station, whereby the dosage of the pharmaceutical composition is administered at the patient monitoring station according to the personalized pharmaceutical delivery system.

The present invention provides a pharmaceutical monitoring and review system that may be used to both monitor and adjust the dosage of a pharmaceutical composition being taken by a patient as well as generate data and information that may be used to enhance the pharmaceutical administration or alter or completely replace the medication with another that is more appropriate. The system comprises a patient monitoring station where the patient is located and which contains a diagnostic means for measuring the patient's physiological condition. The patient monitoring station may optionally contain a personalized pharmaceutical delivery system for the facile measuring of pharmaceutical dosage.

The diagnostic means measures the physiological condition of a patient taking a pharmaceutical composition and generates medical data which is reflective of that condition. The medical data is transmitted by way of an electronic link means to a centralized processing station where the medical data is processed. The centralized processing station is the location of an algorithm and a data storage unit which receives and processes the medical data. The data storage unit has the ability to store historical clinical data on a patient's physiological condition. The algorithm converts the medical data into analyzed data which is interpretable by medical personnel. The analyzed data may be compared and modified in the context of the historical data stored on the data storage unity. In a preferred embodiment the algorithm and the data storage unit will reside in a computer and transfer of information between the two will be mediated by suitable software.

Analyzed data may be interpreted by medical personnel at the centralized processing station and information relating to patient dosage may be relayed to the patient. In one embodiment the analyzed data may be transmitted back to the patient monitoring station via the electronic link means to the diagnostic means. The diagnostic means may be instructed to dispense the appropriate amount of pharmaceutical by interfacing with the personalized pharmaceutical delivery system.

Alternatively, the analyzed data may be transmitted to a data enhancement station. The data enhancement station is the location of a computer which receives analyzed data from a multiplicity of centralized processing stations. The computer processes the analyzed data with input from data generated from clinical trials of related pharmaceuticals and clinical conditions to generate enhanced data. Enhanced data may then be transmitted back to the centralized processing station where it may be review by medical personnel, or it may be transmitted direct to the patient monitoring station. Thus, enhanced data may be used by medical personal to adjust and manage the pharmaceutical dosage at the patient monitoring station, or it may be used to directly affect the dosage without passing through the centralized processing system. Alternatively enhanced data may be transmitted to other data enhancement stations for additional processing and review.

It will be appreciated that monitoring pharmaceutical dosage may be accomplished solely by communication between the patient monitoring station and the centralized processing station without involving the data enhancement station or the generation of enhanced data. Such a system is referred to herein as a pharmaceutical management system and comprises the elements of the patient monitoring station, the diagnostic means, the personalized pharmaceutical delivery system, and the centralized processing station having the algorithm and data storage unit.

### BRIEF DESCRIPTION OF THE FIGURES

FIG. 1 illustrates the general operation of the pharmaceutical dosage management system and the pharmaceutical monitoring and review system for the generation of data by which administration of a pharmaceutical may be managed, at the patient monitoring station.

FIG. 2 illustrates the operation of a pharmaceutical dosage management system for the generation of analyzed data by which the pharmaceutical dosage may be managed.

FIG. 3 illustrates the operation of a pharmaceutical monitoring and review system for the generation of analyzed or enhanced data by which administration of a pharmaceutical may be managed, reviewed or replaced.

### DETAILED DESCRIPTION OF THE PREFERRED EMBODIMENTS

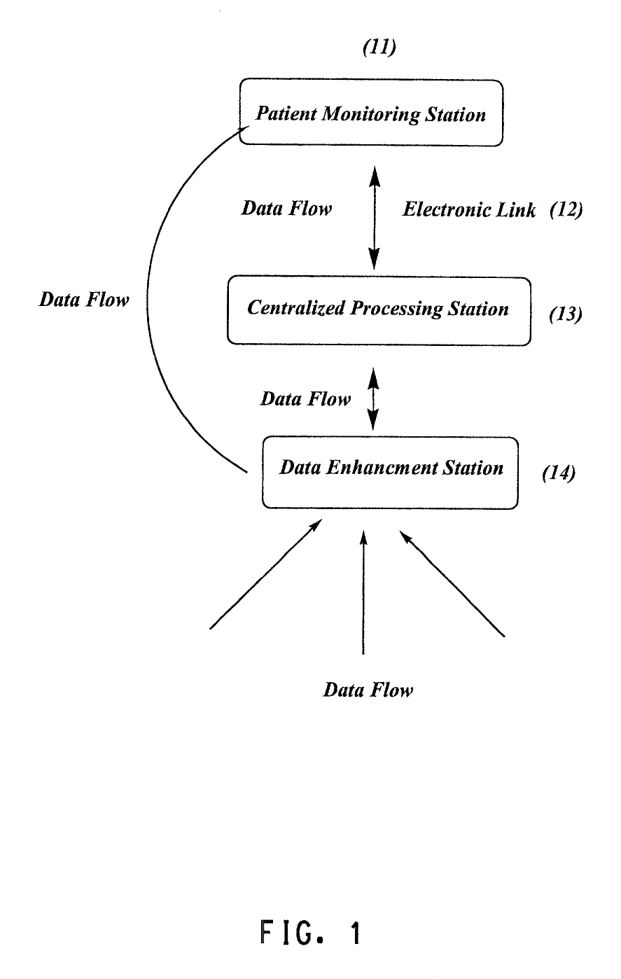

FIG. 1 describes the general data flow between the various elements of the pharmaceutical monitoring and review system. The patient monitoring station (PMS) (11) communicates with the centralized processing station (CPS) (13) via an electronic link means (12). The PMS (11) may be the patient's home, a hospital, a nursing facility, or any other location suitable for monitoring the patient. Data may flow in either direction between the PMS (11) and the CPS (13) depending on the nature of the information. The CPS (13) is the location of medical personnel who will have the expertise to interpret data being transmitted from the PMS (11) to the CPS (13). The electronic means (12) may be any means that is suitable for the transmission of alphanumeric data from one electronic device to another. Suitable means include, but are not limited to, phone lines, fiber optic cable, radio signals, in particular a cellular radio system and satellite communication. Furthermore, suitable means may include a Community Access Television (CATV) network, the Public Switched Telephone Network (PSTN), the Integrated Services Digital Network (ISDN), the Internet, a local area network (LAN), a wide area network (WAN), a wireless communications network, an asynchronous transfer mode (ATM) network or some combination of any of the above.

The CPS (13) communicates with the data enhancement station (DES) (14) where data is received from a multiplicity of CPS. The DES (14) then generates enhanced data which is then communicated either directly to the PMS (11) or to the CPS (13).

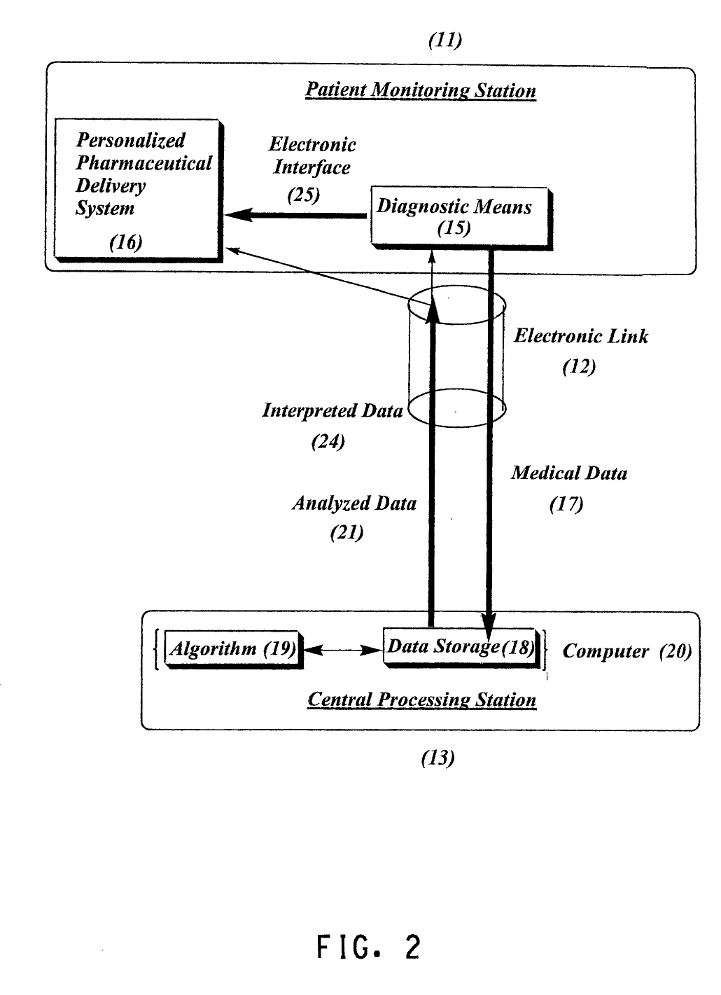

FIG. 2 illustrates the operation of a pharmaceutical management system for the management of a pharmaceutical dosage. The pharmaceutical management system comprises a patient monitoring station (PMS) (11) further comprising a diagnostic means (15) and a personalized pharmaceutical delivery system (PPDS) (16).

The diagnostic means (15) is designed to measure and monitor the physiological condition of a patient during pharmaceutical administration. As used herein “physiological condition” will mean any condition of a patient that is relevant to pharmaceutical dosage. For example, “physiological condition” may mean the levels of pharmaceutical composition in a patient's blood; the levels of breakdown intermediates generated from the pharmaceutical; levels of metabolites that may be altered by the presence or absence of the pharmaceutical (e.g., levels of potassium, sodium, chlorides, blood sugar (glucose), triglycerides, vitamin K and other clotting factors, electrolytes, creatine, urea, nitrogen, protime, amylase, cardiac enzymes, bacterial count, lipid panels, cholesterol, triglycerides, bilirubin, hemoglobin, heparin, uric acid, carcinoembryonic, antigen or other tumor antigens, various reproductive hormones such as those associated with ovulation or pregnancy, carbon dioxide, blood urea nitrogen (BUN), calcium, phosphorus, alkaline phosphatase, lactose dehydrogenase (LDH), serum transaminases (such as SCOT, SGPT, GGT), creatine phosphokinases (CPK), lipase, or amylase); and non-chemical responses to the presence or absence of the pharmaceutical (e.g., blood pressure, heart rate, blood flow, respiration, oxygen saturation, white count, hemoglobin or partial pressure of blood gasses).

The diagnostic means (15) may be any means that converts information reflective of physiological condition to alphanumeric data. Such data is referred to herein as “medical data” (17), which is transmitted via the electronic link means (12) to the CPS (13). The diagnostic means (15) may consist of a standard clinical laboratory test, interfaced with a computer or a home clinical test where the results of the test are converted to alphanumeric data. In a preferred embodiment the diagnostic means (15) is an electronic biosensor capable of monitoring a patient's physiological condition via an electronic sensing means, a chemical means or a combination of both. The diagnostic means (15) may include, but is not limited to, glucose monitoring devices, blood pressure devices, thermometers, pulse oximetry devices, electrocardiograms (EKGs), scales and stethoscopes.

Medical data (17) is received at the CPS (13) by a data storage unit (18). The data storage unit (18) stores historical medical data on the patient's physiological condition. The data storage unit (18) communicates bidirectionally with an algorithm (19) for analyzing the medical data. The algorithm (19) converts the medical data (17) into analyzed data (21). Analyzed data (21) is interpretable by medical personnel and is reflective of the patient's current physiological condition. Analyzed data that is interpreted by medical personnel is referred to herein as “interpreted data” (24). The algorithm may be any computational means whereby medical data may be converted into information that may be interpreted by medical personnel that is reflective of the patients physiological condition. Algorithms may include, but are not limited to, sorting algorithms, tree algorithms, geometric algorithms, graph algorithms and parallel algorithms. In a preferred embodiment the algorithm (19) and the data storage unit (18) are unified in a computer (20).

Analyzed data (21) consists of alphanumeric data that is reflective of a patient's physiologic condition. This data may be transmitted directly back to the PMS (11) or it may be interpreted by medical personnel and converted to interpreted data (24). Both analyzed data (21) and interpreted data (24) may be used at the PMS (11) to manage the pharmaceutical dosage dispensed from the personalized pharmaceutical delivery system (PPDS) (16). In one embodiment analyzed data (21) and interpreted data (24) may be received directly by the PPDS (16) for the dispensing, adjusting and delivery of the pharmaceutical composition. In another embodiment the diagnostic means will be electronically interfaced (25) with the PPDS (16). Analyzed data being received from the CPS (13) will be received directly by the diagnostic means (15) and will be used to instruct the diagnostic means (15) to dispense the appropriate amount of pharmaceutical from the PPDS (16).

The personalized pharmaceutical delivery system (PPDS) (16) is any delivery system which dispenses a pharmaceutical in a formulation comprising a non-toxic, readily digestable, biodegradable pharmaceutical delivery medium; wherein the pharmaceutical is distributed uniformly within the pharmaceutical delivery medium such that division of the delivery medium according to unit length or unit area may be used to quantify the dosage of the pharmaceutical. In a preferred embodiment the pharmaceutical delivery medium is in the form of a tube, or a flat strip. Discrete quantities of the pharmaceutical will be dispensed by cutting a portion of the tube or strip. In one instance the tube or strip may be pre-marked for manual cutting. In another instance the pharmaceutical delivery medium may be contained within a device capable of cutting the tube or strip in accordance with information received from the diagnostic means (15) via the electronic interface (25).

Virtually any pharmaceutical composition may be administered in the instant personalized pharmaceutical delivery system. Suitable pharmaceuticals will include, but not be limited to, any dosage form suitable for oral or rectal administration. Conventional procedures for the selection and preparation of suitable pharmaceutical compositions are described in, for example, Pharmaceutics: The Science of Dosage Form Designs; Aulton, M. E., Ed.; Churchill Livingstone: Melbourne, 1988.

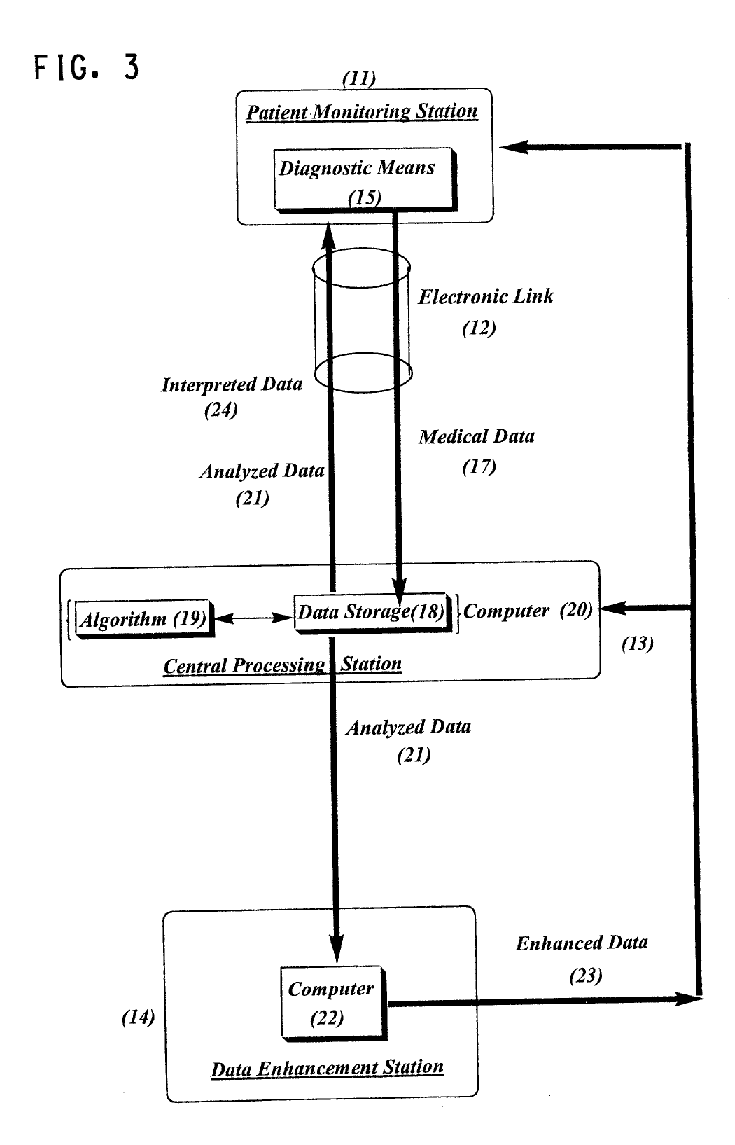

FIG. 3 illustrates a pharmaceutical monitoring and review system for monitoring the administration of a pharmaceutical. The pharmaceutical monitoring and review system comprises many of the same elements as the pharmaceutical management system as described by FIG. 2, including the PMS (11), the diagnostic means (15), the electronic link means (12) for the transmitting of medical data (17), the CPS (13) comprising the data storage unit (18) and the algorithm (19) for the generation of analyzed data (21) or interpreted data (24), all optionally contained within a computer (20). The pharmaceutical monitoring and review system is not dependant on the presence of a personalized pharmaceutical delivery system, although one may optionally be employed at the PMS (11).

The pharmaceutical monitoring and review system is distinguished by the additional component of a data enhancement station (14), comprising a computer (22) for the generation of enhanced data (23). The data enhancement station (DES) (14) may receive analyzed data (21) from a single CPS (13) or a multiplicity of CPS's. The computer (22) of the DES (23) will contain software that will compare all the analyzed data from all sources. Additionally the computer (22) may also contain “prior generated information” relating to clinical trials of a pharmaceutical, research studies on the active or inactive components in the pharmaceutical, and information relating to the organic condition that the pharmaceutical attempts to treat. The computer (22) will generate enhanced data (23) that will contain additional information about the pharmaceutical that is not present in the analyzed data (21). Enhanced data (23) may be used independently of the pharmaceutical monitoring and review system, or may be transmitted to the CPS (13) and converted to interpreted data (24), or may be transmitted directly to the PMS (11) or the diagnostic means (15). Enhanced data may be used to alter the dosage of the pharmaceutical composition (including stopping the dose), may be used to suggest the use of additional pharmaceutical compositions or may be used to suggest replacing the composition currently in use with another.

What is claimed is:

1. A pharmaceutical monitoring and review system for transmitting medical data between a patient monitoring station and a central processing station and between the central processing station and one or more data enhancement stations over an integrated network the monitoring and review system comprising:
a) a patient monitoring station comprising a diagnostic means for measuring patient physiological condition in response to the presence of at least one pharmaceutical composition, the diagnostic means collecting and transmitting medical data via an electronic link means to a central processing station;
b) a central processing station for receiving medical data from the diagnostic means; the central processing station comprising an algorithm for analysis of the medical data and a data storage unit for the storage and retrieval of medical data; the central processing station transmitting the analyzed data to the patient monitoring station and optionally to at least one data enhancement station; and
c) a data enhancement station comprising at least one computer to receive the analyzed medical data from one or more central processing stations; the computer comparing the analyzed medical data received from the one or more central processing stations with prior generated data to generate enhanced data.

2. A pharmaceutical monitoring and review system according to claim 1 wherein the at least one pharmaceutical composition is contained in a personalized pharmaceutical delivery system.

3. A pharmaceutical monitoring and review system according to claim 2 wherein the personalized pharmaceutical delivery system comprises:
a) a pharmaceutical; and
b) a non-toxic, biodegradable pharmaceutical delivery medium; wherein the pharmaceutical is distributed uniformly within the pharmaceutical delivery medium such that division of the delivery medium according to unit length or unit area may be used to quantify the dosage of the pharmaceutical.

4. A pharmaceutical monitoring and review system according to claim 1 wherein the physiological condition is characterized by measurements in a patient selected from the group consisting of levels of pharmaceutical, levels of pharmaceutical intermediates, levels of physiological metabolites, and nonchemical responses to the presence or absence of a pharmaceutical

5. A pharmaceutical monitoring and review system according to claim 1 wherein the medical data is an alphanumeric or digital representation of the physiological condition.

6. A pharmaceutical monitoring and review system according to claim 1 wherein the diagnostic means is selected from the group consisting of electronic biosensors, a clinical laboratory test and a home clinical test, the diagnostic means generating alphanumeric or digital medical data.

7. A pharmaceutical monitoring and review system according to claim 1 wherein the electronic link means is selected from the group consisting of a fiberoptic cable and a radio signal.

8. A pharmaceutical monitoring and review system according to claim 1 wherein the algorithm and the data storage unit are contained in a computer.

9. A pharmaceutical monitoring and review system according to claim 1 wherein the pharmaceutical composition is packaged in discrete amounts.

10. A pharmaceutical monitoring and review system according to claim 1 wherein enhanced data comprises medical data and analyzed data from a multiplicity of different centralized processing stations and patient monitoring means.

11. A method of monitoring and reviewing the administration of a pharmaceutical composition by operating a pharmaceutical monitoring and review system of claim 1 comprising:
a) collecting medical data in a diagnostic means at the patient monitoring station, the data reflective of a patient's physiological condition in response to the presence of at least one pharmaceutical composition;
b) transmitting the medical data from the diagnostic means via an electronic link means to a central processing station comprising an algorithm and a data storage unit for data analysis;
c) analyzing the medical data by the algorithm to generate analyzed data;
d) transmitting the analyzed data to least one computer located at a data enhancement station and to the patient monitoring station, whereby the dosage of the pharmaceutical composition is administered at the patient monitoring station;
e) collecting analyzed data from one or more central processing stations in the computer at the data enhancement station whereby enhanced data is generated; and
f) transmitting some or all of the enhanced data generated by the at least one computer to either or both of the patient monitoring station and the central processing station whereby dosage of the pharmaceutical composition is administered at the patient monitoring station.

12. A method according to claim 11 wherein the at least one pharmaceutical composition is contained in a personalized pharmaceutical delivery system.

13. A method according to claim 12 wherein the personalized pharmaceutical delivery system comprises:
a) a pharmaceutical; and
b) a non-toxic, biodegradable pharmaceutical delivery medium; wherein the pharmaceutical is distributed uniformly within the pharmaceutical delivery medium such that division of the delivery medium according to unit length or unit area may be used to quantify the dosage of the pharmaceutical.

14. A method according to claim 11 wherein the algorithm and the data storage unit are contained in a computer.

15. A method according to claim 11 wherein after step (c) the analyzed data is converted to interpreted data.

16. A pharmaceutical management system for transmitting medical data between a patient monitoring station and a central processing station over an integrated network the management system comprising:
a) a patient monitoring station comprising a diagnostic means for measuring patient physiological condition in response to the presence of at least one pharmaceutical composition; the composition contained in a personalized pharmaceutical delivery system, the diagnostic means collecting and transmitting medical data via an electronic link means to a central processing station;
b) a central processing station for receiving medical data from the diagnostic means; the central processing station comprising an algorithm and a data storage unit for storage and analysis of the medical data; the central processing station transmitting the analyzed medical data to the patient monitoring station whereby the at least one pharmaceutical composition is administered according to the personalized pharmaceutical delivery system.

17. A pharmaceutical management system according to claim 16 wherein the personalized pharmaceutical delivery system comprises:
a) a pharmaceutical; and
b) a non-toxic, biodegradable pharmaceutical delivery medium; wherein the pharmaceutical is distributed uniformly within the pharmaceutical delivery medium such that division of the delivery medium according to unit length or unit area may be used to quantify the dosage of the pharmaceutical.

18. A pharmaceutical management system according to claim 16 wherein the physiological condition is characterized by measurements in a patient selected from the group consisting of levels of pharmaceutical, levels of pharmaceutical intermediates, levels of physiological metabolites, and non-chemical responses to the presence or absence of a pharmaceutical.

19. A pharmaceutical management system according to claim 16 wherein the medical data is an alphanumeric or digital representation of the physiological condition.

20. A pharmaceutical management system according to claim 16 wherein the diagnostic means is selected from the group consisting of electronic biosensors, a clinical laboratory test and a home clinical test, the diagnostic means generating alphanumeric or digital medical data.

21. A pharmaceutical management system according to claim 16 wherein the diagnostic means is interfaced with the personalized pharmaceutical delivery system.

22. A pharmaceutical management system according to claim 16 wherein the electronic link means is selected from the group consisting of a fiber-optic cable and a radio signal.

23. A pharmaceutical management system according to claim 16 wherein the algorithm and the data storage unit are contained in a computer.

24. A pharmaceutical management system according to claim 16 wherein the pharmaceutical composition is packaged in discrete amounts.

25. A method for managing pharmaceutical dosage of a pharmaceutical contained in a personalized pharmaceutical delivery system by operating the pharmaceutical management system of claim 16 comprising:
a) collecting medical data in a diagnostic means at the patient monitoring station, the data reflective of a patient's physiological condition in response to the presence of at least one pharmaceutical composition contained within a personalized pharmaceutical delivery system;
b) transmitting the medical data from the diagnostic means via an electronic link means to a central processing station comprising an algorithm and a data storage unit for data storage and analysis;
c) analyzing the medical data by the algorithm to generate analyzed data; and
d) transmitting the analyzed data to the patient monitoring station, whereby the dosage of the pharmaceutical composition is administered at the patient monitoring station according to the personalized pharmaceutical delivery system.

26. A method according to claim 25 wherein the personalized pharmaceutical delivery system comprises:
a) a pharmaceutical; and
b) a non-toxic, biodegradable pharmaceutical delivery medium; wherein the pharmaceutical is distributed uniformly within the pharmaceutical delivery medium such that division of the delivery medium according to unit length or unit area may be used to quantify the dosage of the pharmaceutical.

27. A method according to claim 25 wherein the algorithm and the data storage unit are contained in a computer.

28. A method according to claim 25 wherein the algorithm and the data storage unit are contained in a computer.

29. A method according to claim 25 wherein the diagnostic means is interfaced with the personalized pharmaceutical delivery system.

30. A personalized pharmaceutical delivery system comprising:
a) a pharmaceutical; and
b) a non-toxic, biodegradable pharmaceutical delivery medium; wherein the pharmaceutical is distributed uniformly within the pharmaceutical delivery medium such that division of the delivery medium according to unit length or unit area may be used to quantify the dosage of the pharmaceutical.

31. A pharmaceutical delivery system according to claim 30 wherein the pharmaceutical delivery medium is in the form selected from the group consisting of a tube and a flat strip.
